(19) United States
(12) Patent Application Publication
(10) Pub. No.: US 2006/0094714 A1
(43) Pub. Date: May 4, 2006

(54) Compounds and their use to treat diabetes and related disorders

(75) Inventors: William H Bullock, Easton, CT (US); Qingjie Liu, Orange, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/543,070

(22) PCT Filed: Mar. 26, 2004
(86) PCT No.: PCT/US04/09342

§ 371 Date: Jul. 22, 2005

(51) Int. Cl. A61K 31/5415; A61K 31/506; A61K 31/5377; A61K 31/496; A61K 31/501; A61K 31/4745; C07D 471/02

(52) U.S. Cl. 514/227.8; 514/232.5; 514/252.04; 514/256; 514/253.04; 514/300; 546/122; 544/60; 544/127; 544/238; 514/362

(57) ABSTRACT

The invention relates generally to compounds described herein. More specifically, the invention relates to compounds and pharmaceutical compositions containing such compounds. Methods of the invention comprise administration of a compound of the invention for the treatment of diabetes and related disorders.

This application claims benefit of U.S. Provisional Application Ser. No. 60/455,194, Mar. 26, 2003, the contents of which are incorporated herein by reference in their entirety.

### FIELD OF THE INVENTION

The present invention relates to compounds, pharmaceutical compositions containing them, and their use for treating diabetes and related disorders in a subject.

### DESCRIPTION OF THE RELATED ART

Diabetes is characterized by impaired glucose metabolism manifesting itself among other things by an elevated blood glucose level in the diabetic patient. Underlying defects lead to a classification of diabetes into two major groups. Type 1 diabetes, or insulin dependent diabetes mellitus (IDDM), arises when patients lack insulin-producing β-cells in their pancreatic glands. Type 2 diabetes, or non-insulin dependent diabetes mellitus (NIDDM), occurs in patients with impaired β-cell function and alterations in insulin action.

The current treatment for type 1 diabetic patients is the injection of insulin, while the majority of type 2 diabetic patients are treated with agents that stimulate β-cell function or with agents that enhance the tissue sensitivity of the patients towards insulin. The drugs presently used to treat type 2 diabetes include alpha-glucosidase inhibitors, insulin sensitizers, insulin secretagogues, and metformin.

Over time, almost one-half of type 2 diabetic subjects lose their response to these agents. Insulin treatment is instituted after diet, exercise, and oral medications have failed to adequately control blood glucose. The drawbacks of insulin treatment are the need for drug injection, the potential for hypoglycemia, and weight gain.

Because of the problems with current treatments, new therapies to treat type 2 diabetes are needed. In particular, new treatments to retain normal (glucose-dependent) insulin secretion are needed. Such new drugs should have the following characteristics: dependency on glucose for promoting insulin secretion, i.e., compounds that stimulate insulin secretion only in the presence of elevated blood glucose; low primary and secondary failure rates; and preservation of islet cell function. The strategy to develop the new therapy disclosed herein is based on the cyclic adenosine monophosphate (cAMP) signaling mechanism and its effects on insulin secretion.

Metabolism of glucose promotes the closure of ATP-dependent K+ channels, which leads to cell depolarization and subsequent opening of Ca++ channels. This in turn results in the exocytosis of insulin granules. cAMP is a major regulator of glucose-stimulated insulin secretion. However, it has little if any effects on insulin secretion in the absence of or at low glucose concentrations (Weinhaus, et al., Diabetes 47:1426-1435, 1998). The effects of cAMP on insulin secretion are thought to be mediated by a protein kinase A pathway.

Endogenous secretagogues like pituitary adenylate cyclase activating peptide (PACAP), VIP, and GLP-1 use the cAMP system to regulate insulin secretion in a glucose-dependent fashion (Komatsu, et al., Diabetes 46:1928-1938, 1997). Also, phosphodiesterases (PDEs) are known to be involved in the regulation of the cAMP system.

PACAP is a potent stimulator of glucose-dependent insulin secretion from pancreatic β-cells. Three different PACAP receptor types (R1, R2, and R3) have been described (Harmar, et al., Pharmacol. Rev. 50:265-270, 1998). The insulinotropic action of PACAP is mediated by the GTP binding protein Gs. Accumulation of intracellular cAMP in turn activates nonselective cation channels in β-cells increasing [Ca++]i, and promoting the exocytosis of insulin-containing secretory granules.

Vasoactive intestinal peptide (VIP) is a 28 amino acid peptide that was first isolated from hog upper small intestine (Said and Mutt, Science 169:1217-1218, 1970; U.S. Pat. No. 3,879,371). This peptide belongs to a family of structurally related, small polypeptides that includes helodermin, secretin, the somatostatins, and glucagon. The biological effects of VIP are mediated by the activation of membrane-bound receptor proteins that are coupled to the intracellular cAMP signaling system. These receptors were originally known as VIP-R1 and VIP-R2, however, they were later found to be the same receptors as PACAP-R2 and PACAP-R3.

GLP-1 is released from the intestinal L-cell after a meal and functions as an incretin hormone (i.e., it potentiates glucose-induced insulin release from the pancreatic β-cell). It is a 37-amino acid peptide that is differentially expressed by the glucagon gene, depending upon tissue type. The clinical data that support the beneficial effect of raising cAMP levels in β-cells have been collected with GLP-1. Infusions of GLP-1 in poorly controlled type 2 diabetics normalized their fasting blood glucose levels (Gutniak, et al., New Eng. J. Med. 326:1316-1322, 1992) and with longer infusions improved the β-cell function to those of normal subjects (Rachman, et al., Diabetes 45:1524-1530, 1996). A recent report has shown that GLP-1 improves the ability of β-cells to respond to glucose in subjects with impaired glucose tolerance (Byrne, et al., Diabetes 47:1259-1265, 1998). All of these effects, however, are short-lived because of the short half-life of the peptide.

1-(3-Chlorophenyl)-4-(dimethylamino)-1,8-naphthyridin-2(1H)-one is known from JP 07126268 as a precursor for 3-(1-hydroxyalkyl)-4-(dimethylamino)-1,8-naphthyridin-2(1H)-ones. This application describes a number of naphthyridine derivatives as inflammation inhibitors. Kuge, et al., (Synthetic Communications 24(22):3289-96, 1994) describe 4-amino-1-phenyl-1,8-Naphthyridin-2(1H)-one as a precursor to prepare 5-phenylimidazo[4,5-c][1,8]naphthyridin-4(5H)-one, which exhibits potent antiasthmatic activity.

### SUMMARY OF THE INVENTION

The invention provides compounds, pharmaceutical compositions containing them, and methods of using the same for treating diabetes and related disorders. Compounds of the invention include compounds shown below in the section entitled “Detailed Description of the Invention.”

The present invention relates to a compound of the formula 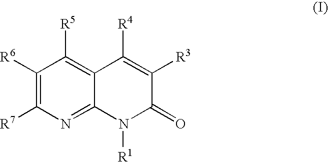
wherein

R¹ is selected from alkyl of 1-8 carbon atoms, alkenyl of 2-8 carbon atoms, alkynyl of 2-8 carbon atoms, and A-R⁹,
or
R¹ is selected from aryl of 6-10 carbon atoms, heteroaryl of 2-9 carbon atoms and 1-4 heteroatoms selected from N, S(═O)₀₋₂ and O, cycloalkyl of 3-8 carbon atoms, cycloalkenyl of 4-8 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), all of which may be substituted with 1-3 of R¹⁰;
   R¹⁰ is selected from nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms, haloalkoxy of 1-6 carbon atoms, cycloalkoxy of 3-6 carbon atoms, aryl of 6-10 carbon atoms, heteroaryl of 2-9 carbon atoms and 1-4 heteroatoms selected from N, S(═O)₀₋₂ and O, NR¹¹R¹², C(═O)O¹¹, C(═O)NHR¹¹, NHC(═O)R¹³, NHS(═O)₂R¹³, S(═O)₀₋₂R¹³, S(═O)₂NHR¹¹, cycloalkyl of 3-6 carbon atoms, cycloalkenyl of 3-6 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O);
      R¹³ is selected from alkyl of 1-6 carbon atoms, alkenyl of 2-6 carbon atoms, alkynyl of 2-6 carbon atoms, haloalkyl of 1-6 carbon atoms, cycloalkyl of 3-6 carbon atoms, and cycloalkenyl of 4-6 carbon atoms;
      R¹¹ and R¹² are independently selected from hydrogen, alkyl of 1-6 carbon atoms, alkenyl of 2-6 carbon atoms, alkynyl of 2-6 carbon atoms, haloalkyl of 1-6 carbon atoms, cycloalkyl of 3-6 carbon atoms, and cycloalkenyl of 4-6 carbon atoms;
   A is selected from alkyl of 1-8 carbon atoms, alkenyl of 2-8 carbon atoms, alkynyl of 2-8 carbon atoms, and haloalkyl of 1-8 carbon atoms;
   R⁹ is selected from hydroxy, alkoxy of 1-6 carbon atoms, cycloalkoxy of 3-6 carbon atoms, O-A-R¹⁴, NR¹¹R¹²; or
   R⁹ is selected from aryl of 6-10 carbon atoms, heteroaryl of 2-9 carbon atoms and 1-4 heteroatoms selected from N, S(═O)₀₋₂ and O, cylcoalkyl of 3-8 carbon atoms, cycloalkenyl of 5-8 carbon atoms, all of which may be substituted with 1-3 of R¹⁰, or
   R⁹ is selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
      R¹⁴ is selected from cylcoalkyl of 3-8 carbon atoms, cycloalkenyl of 5-8 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroatoms selected from N, S(═O)₀₋₂ and O, all of which may be substituted with 1-3 of R¹⁰;
R³ is hydrogen,
R⁴ is —NR⁴⁻¹R⁴⁻²;
   R⁴⁻¹ is selected from the group consisting of hydrogen, alkyl of 1-8 carbon atoms, alkenyl of 2-8 carbon atoms, alkynyl of 2-8 carbon atoms and haloalkyl of 1-8 carbon atoms;
   R⁴⁻² is selected from the group consisting of hydrogen, alkyl of 1-8 carbon atoms, alkenyl of 2-8 carbon atoms, alkynyl of 2-8 carbon atoms, haloalkyl of 1-8 carbon atoms, aryl of 6-10 carbon atoms, heteroaryl of 2-9 carbon atoms and 1-4 heteroatoms, 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said aryl, hereroaryl, heterocycloalkyl or heterocycloalkenyl may be substituted with one to three substituents selected from the group consisting of nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms and haloalkoxy of 1-6 carbon atoms, or
   R⁴⁻¹ and R⁴⁻² form a 5-7 membered heterocycloalkyl of 3-6 carbon atoms and 1-2 heteroatoms selected from N, S(═O)₀₋₂ and O, wherein said heterocycloalkyl may be substituted with one to three substituents selected from the group consisting of nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms and haloalkoxy of 1-6 carbon atoms,
R⁵ and R⁶ are independently selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms, all of which may be substituted with 1-3 of R¹⁰,
or
R⁵ and R⁶ are independently selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, A-R²³, A-NR²⁴R²⁵, C(═O)R²⁴, C(═O)OR²⁴, C(═O)NR²⁴R²⁵, S(═O)₂R²⁶, A-C(═O)OR²⁴, or A-C(═O)NR²⁴R²⁵,
or
R⁵ and R⁶ are independently selected from hydrogen, halogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
   Q is selected from O and S(═O)₀₋₂;
   R²² is selected from hydrogen, alkyl of 1-8 carbon atoms, haloalkyl of 1-8 carbon atoms, and cycloalkyl of 3-8 carbon atoms;
   R²³ is selected from hydroxy, alkoxy of 1-8 carbon atoms, haloalkoxy of 1-8 carbon atoms, and cycloalkoxy of 3-8 carbon atoms, or
   R²³ is selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
   R²³ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
      with the proviso for A(OR²²)—R²³ that when R²³ is selected from hydroxy, alkoxy of 1-8 carbon atoms, haloalkoxy of 1-8 carbon atoms, and cycloalkoxy of 3-8 carbon atoms, A is not CH;
   R²⁴ and R²⁵ are independently selected from hydrogen, alkyl of 1-6 carbon atoms, alkenyl of 2-6 carbon atoms, alkynyl of 2-6 carbon atoms, haloalkyl of 1-6 carbon atoms, and A-R²³, or
   R²⁴ and R²⁵ are independently selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
   R²⁴ and R²⁵ are independently selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, or
   R²⁴ and R²⁵ combine, together with the nitrogen atom to which they are attached, to form a 5-7 membered heterocycloalkyl of 3-6 carbon atoms and 1-2 heteroatoms selected from N, S(═O)₀₋₂, and O, a 5-7 membered heterocycloalkenyl of 3-6 carbon atoms and 1-2 heteroatoms selected from N, S(═O)₀₋₂, and O, or a heteroaryl of 2-9 carbon atoms and 1-4 heteroatoms, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), all of which may be substituted with 1-3 of R¹⁰;
   R²⁶ is selected from alkyl of 1-6 carbon atoms, alkenyl of 2-6 carbon atoms, alkynyl of 2-6 carbon atoms, haloalkyl of 1-6 carbon atoms, A(OR²²)—R²³, and A-R²³, or
   R²⁶ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
   R²⁶ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
   R²⁷ is selected from hydrogen, alkyl of 1-6 carbon atoms, alkenyl of 2-6 carbon atoms, alkynyl of 2-6 carbon atoms, haloalkyl of 1-6 carbon atoms, and A-R²³, or
   R²⁷ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
   R²⁷ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
   R²⁸ is selected from hydrogen, alkyl of 1-6 carbon atoms, alkenyl of 2-6 carbon atoms, alkynyl of 2-6 carbon atoms, haloalkyl of 1-6 carbon atoms, A-R²³, C(═O)R²⁴, C(═O)OR²⁶, C(═O)NR²⁵R³⁰, S(═O)₂R²⁶, A-C(═O)R²⁴, A-C(═O)OR²⁴, and A-C(═O)NR²⁴R²⁵, or
   R²⁸ is selected from cycloalkyl of 3-6 carbon atoms, cycloalkenyl of 3-6 carbon atoms, aryl of 6-10 carbon atoms, heteroaryl of 2-9 carbon atoms and 1-4 heteroatoms selected from N, S(═O)₀₋₂, and O, all of which may be substituted with 1-3 of R¹⁰, or
   R²⁸ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
   R³⁰ is selected from alkyl of 1-6 carbon atoms, alkenyl of 2-6 carbon atoms, alkynyl of 2-6 carbon atoms, haloalkyl of 1-6 carbon atoms, A(OR²²)—R²³, and A-R²³, or
   R³⁰ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
   R³⁰ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, or
   R²⁵ and R³⁰ combine, together with the nitrogen atom to which they are attached, to form a 5-7 membered heterocycloalkyl of 3-6 carbon atoms and 1-2 heteroatoms selected from N, S(═O)₀₋₂, and O, a 5-7 membered heterocycloalkenyl of 3-6 carbon atoms and 1-2 heteroatoms selected from N, S(═O)₀₋₂, and O, or a heteroaryl of 2-9 carbon atoms and 1-4 heteroatoms, all of which may be substituted with 1-3 of R¹⁰;
   R²⁹ is selected from alkyl of 1-6 carbon atoms, alkenyl of 2-6 carbon atoms, alkynyl of 2-6 carbon atoms, haloalkyl of 1-6 carbon atoms, A-R²³, A-C(═O)R²⁴, A-C(═O)OR²⁴, A-C(═O)NR²⁴R²⁵, A-NR²⁷R²⁸, or
   R²⁹ is selected from cycloalkyl of 3-6 carbon atoms, cycloalkenyl of 3-6 carbon atoms, aryl of 6-10 carbon atoms, heteroaryl of 2-9 carbon atoms and 1-4 heteroatoms selected from N, S(═O)₀₋₂, and O, all of which may be substituted with 1-3 of R¹⁰, or
   R²⁹ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R⁷ is selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms, all of which may be substituted with 1-3 of R¹⁰,
or
R⁷ is selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, A(OR²²)—R²³, A-R²³, A-NR²⁴R²⁵, C(═O)R²⁴, C(═O)OR²⁴, C(═O)NR²⁴R²⁵, S(═O)₂R²⁶, A-C(═O)R²⁴, A-C(═O)OR²⁴, or A-C(═O)NR²⁴R²⁵,
or
R⁷ is selected from hydrogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
and a pharmaceutically acceptable salt thereof,
   with the proviso that the compound is not 1-(3-chlorophenyl)-4dimethylamino)-1,8-naphthyridin-2(1H)-one or 4-amino-1-phenyl-1,8-naphthyridin-2(1H)-one.

In another embodiment, the present invention relates to a compound of the formula (I), wherein
R¹ is selected from aryl of 6 or 10 carbon atoms, which may be substituted with 1-3 of R¹⁰;
   R¹⁰ is selected from nitro, nitrile, hydroxy, halogen, acyl of 1-6 carbon atoms, alkyl of 1-6 carbon atoms, haloalkyl of 1-6 carbon atoms, alkoxy of 1-6 carbon atoms, haloalkoxy of 1-6 carbon atoms, cycloalkoxy of 3-6 carbon atoms, phenyl, heteroaryl selected from thienyl, furyl, pyrrolyl, thiazolyl, oxazolyl, imidazolyl, pyridyl, pyrimidyl, pyridazinyl, indolyl, indazolyl, benzofurayl, benzothiophenyl, quinolinyl and isoquinolinyl, NR¹¹R¹², C(═O)OR¹¹, C(═O)NHR¹¹, NHC(═O)R¹³, NHS(═O)₂R¹³, S(═O)₀₋₂R¹³, S(═O)₂NHR¹¹, cyclopropyl, cyclopentyl, cyclohexyl, and heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl;
   R¹³ is selected from alkyl of 1-6 carbon atoms, haloalkyl of 1-6 carbon atoms, and cycloalkyl of 3-6 carbon atoms;
   R¹¹ and R¹² are independently selected from hydrogen, alkyl of 1-6 carbon atoms, haloalkyl of 1-6 carbon atoms, and cycloalkyl of 3-6 carbon atoms;
   A is selected from alkyl of 1-8 carbon atoms and haloalkyl of 1-8 carbon atoms;
R³ is hydrogen,
R⁴ is —NR⁴⁻¹R⁴⁻²;
   R⁴⁻¹ is selected from the group consisting of hydrogen, alkyl of 1-8 carbon atoms and haloalkyl of 1-8 carbon atoms;
   R⁴⁻² is selected from the group consisting of hydrogen, alkyl of 1-8 carbon atoms, haloalkyl of 1-8 carbon atoms, aryl of 6 or 10 carbon atoms, heteroaryl selected from thienyl, furyl, pyrrolyl, thiazolyl, oxazolyl, imnidazolyl, pyridyl, pyrimidyl, pyridazinyl, indolyl, indazolyl, benzofuranyl, benzothiophenyl, quinolinyl, isoquinolinyl, heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said aryl, hereroaryl or heterocycloalkyl may be substituted with one to three substituents selected from the group consisting of nitro, nitrile, hydroxy, halogen, acyl of 1-6 carbon atoms, alkyl of 1-6 carbon atoms, haloalkyl of 1-6 carbon atoms, alkoxy of 1-6 carbon atoms and haloalkoxy of 1-6 carbon atoms, or
   R⁴⁻¹ and R⁴⁻² form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may be substituted with one to three substituents selected from the group consisting of nitro, nitrile, hydroxy, halogen, acyl of 1-6 carbon atoms, alkyl of 1-6 carbon atoms, haloalkyl of 1-6 carbon atoms, alkoxy of 1-6 carbon atoms and haloalkoxy of 1-6 carbon atoms,
R⁵ and R⁶ are independently selected from cycloalkyl of 3-8 carbon atoms, aryl of 6-10 carbon atoms, and heteroaryl of 2-9 carbon atoms and 1-4 heteroatoms, all of which may be substituted with 1-3 of R¹⁰,
or
R⁵ and R⁶ are heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl,
or
R⁵ and R⁶ are independently selected from hydrogen, halogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, N²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
   Q is selected from O and S(═O)₀₋₂;
   R²² is selected from hydrogen, alkyl of 1-8 carbon atoms, haloalkyl of 1-8 carbon atoms, and cycloalkyl of 3-8 carbon atoms;
   R²³ is selected from hydroxy, alkoxy of 1-8 carbon atoms, haloalkoxy of 1-8 carbon atoms, and cycloalkoxy of 3-8 carbon atoms, or
   R²³ is selected from cycloalkyl of 3-8 carbon atoms, aryl of 6 or 10 carbon atoms, and heteroaryl of 2-9 carbon atoms and 1-4 heteroatoms selected from N, S(═O)₀₋₂, and O, all of which may be substituted with 1-3 of R¹⁰, or
   R²³ is heterocycloalkyl selected from tetrahydrofuryl, pyrrolidinyl, pyrrolinyl, piperidinyl, 1,2-dihydropyridinyl, 1,4dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
   with the proviso for A(OR²²)—R²³ that when R²³ is selected from hydroxy, alkoxy of 1-8 carbon atoms, haloalkoxy of 1-8 carbon atoms, and cycloalkoxy of 3-8 carbon atoms, A is not CH;
   R²⁴ and R²⁵ are independently selected from hydrogen, alkyl of 1-6 carbon atoms, haloalkyl of 1-6 carbon atoms, and A-R²³, or
   R²⁴ and R²⁵ are independently selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl, all of which may be substituted with 1-3 of R¹⁰, or
   R²⁴ and R²⁵ are heterocycloalkyl selected from tetrahydrofuryl, pyrrolidinyl, pyrrolinyl, piperidinyl, 1,2dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰, or
   R²⁴ and R²⁵ combine, together with the nitrogen atom to which they are attached, to form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, all of which may be substituted with 1-3 of R¹⁰;
   R²⁶ is selected from alkyl of 1-6 carbon atoms, haloalkyl of 1-6 carbon atoms, A(OR²²)—R²³, and A-R²³, or
   R²⁶ is selected from cyclopropyl, cyclopentyl, cyclohexyl, aryl of 6 or 10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl, all of which may be substituted with 1-3 of R¹⁰, or
   R²⁶ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
   R²⁷ is selected from hydrogen, alkyl of 1-6 carbon atoms, haloalkyl of 1-6 carbon atoms, and A-R²³, or
   R²⁷ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofturanyl, benzothiophenyl, quinolinyl, isoquinolinyl, all of which may be substituted with 1-3 of R¹⁰, or
   R²⁷ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
   R²⁸ is selected from hydrogen, alkyl of 1-6 carbon atoms, haloalkyl of 1-6 carbon atoms, A-R²³, C(═O)R²⁴, C(═O)OR²⁶, C(═O)NR²⁵R³⁰, S(═O)₂R²⁶, A-C(═O)R²⁴, A-C(═O)OR²⁴, and A-C(═O)NR²⁴R²⁵, or
   R²⁸ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰, or
   R²⁸ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
   R³⁰ is selected from alkyl of 1-6 carbon atoms, haloalkyl of 1-6 carbon atoms, A(OR²²)—R²³, and A-R²³, or
   R³⁰ is selected from cyclopropyl, cyclopentyl, cyclohexyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰, or
   R³⁰ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰, or
   R²⁵ and R³⁰ combine, together with the nitrogen atom to which they are attached, to form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide, thiomorpholinyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, or isoquinolinyl, all of which may be substituted with 1-3 of R¹⁰;
   R²⁹ is selected from alkyl of 1-6 carbon atoms, haloalkyl of 1-6 carbon atoms, A-R²³, A-C(═O)R²⁴, A-C(═O)OR²⁴, A-C(═O)NR²⁴R²⁵, A-NR²⁷R²⁸, or
   R²⁹ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, all of which may be substituted with 1-3 of R¹⁰, or
   R²⁹ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
R⁷ is selected from cycloalkyl of 3-8 carbon atoms, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰,
or
R⁷ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, and/or wherein one or more of the carbon atoms in said heterocycloalkyl may be oxidized to C(═O), wherein said heterocycloalkyl may be substituted with 1-3 of R¹⁰, A(OR²²)—R²³, A-R²³, A-NR²⁴R²⁵, C(═O)R²⁴, C(═O)OR²⁴, C(═O)NR²⁴R²⁵, S(═O)₂R²⁶, A-C(═O)R²⁴, A-C(═O)OR²⁴, or A-C(═O)NR²⁴R²⁵,
or
R⁷ is selected from hydrogen, nitrile, nitro, hydroxy, alkyl of 1-8 carbon atoms, haloalkyl of 1-8 carbon atoms, alkoxy of 1-8 carbon atoms, haloalkoxy of 1-8 carbon atoms, cycloalkoxy of 3-8 carbon atoms, AR²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
and pharmaceutically acceptable salts thereof,
   with the proviso that the compound is not 1-(3-chlorophenyl)-4-(dimethylamino)-1,8-naphthyridin-2(1H)-one or 4-amino-1-phenyl-1,8-naphthyridin-2(1H)-one.

In another embodiment, the present invention relates to a compound of the formula (I), wherein
R¹ is phenyl, which may be substituted with 1-3 of R¹⁰;
   R¹⁰ is selected from nitro, nitrile, hydroxy, halogen, trifluoromethyl, methylcarbonyl, ethylcarbonyl, methyl, ethyl, propyl, isopropyl, butyl, t-butyl, methoxy, ethoxy, propyloxy or isopropyloxy;
R³ is hydrogen,
R⁴ is —NR⁴⁻¹R⁴⁻²;
   R⁴⁻¹ is selected from the group consisting of hydrogen, methyl, ethyl, propyl, isopropyl, butyl and t-butyl;
   R⁴⁻² is phenyl, wherein said phenyl may be substituted with one to three substituents selected from the group consisting of nitro, nitrile, hydroxy, fluoro, chloro, methylcarbonyl, ethylcarbonyl, methyl, ethyl, propyl, isopropyl, butyl, t-butyl, methoxy, ethoxy, propyloxy or isopropyloxy; or
   R⁴⁻¹ and R⁴⁻² form a heterocycloalkyl selected from piperazinyl, morpholinyl and thiomorpholinyl, wherein said heterocycloalkyl may be substituted with one to three substituents selected from the group consisting of nitro, nitrile, hydroxy, fluoro, chloro, methylcarbonyl, ethylcarbonyl, methyl, ethyl, propyl, isopropyl, butyl, t-butyl, methoxy, ethoxy, propyloxy and isopropyloxy,
R⁵ and R⁶ are independently selected from hydrogen, fluoro, chloro, nitrile, nitro, hydroxy, methyl, ethyl, propyl, isopropyl, butyl, t-butyl, trifluoromethyl, methoxy, ethoxy, propyloxy and isopropyloxy;
R⁷ is selected from hydrogen, nitrile, nitro, hydroxy, methyl, ethyl, propyl, isopropyl, butyl, t-butyl, trifluoromethyl, methoxy, ethoxy, propyloxy and isopropyloxy;
and pharmaceutically acceptable salts thereof,
   with the proviso that the compound is not 1-(3-chlorophenyl)-4-(dimethylamino)-1,8-naphthyridin-2(1H)-one or 4-amino-1-phenyl-1,8-naphthyridin-2(1H)-one.

In another embodiment, the present invention relates to a compound of the formula (I), wherein
R¹ is phenyl, which may be substituted with 1-3 of R¹⁰;
R¹⁰ is selected from fluoro, chloro and trifluoromethyl;
R³ is hydrogen,
R⁴ is —NR⁴⁻¹R⁴⁻²;
R⁴⁻¹ is selected from the group consisting of hydrogen and methyl;
R⁴⁻² is phenyl, wherein said phenyl may be substituted with one or two substituents selected from the group consisting of nitrite, fluoro, chloro, methyl, ethyl, methoxy and ethoxy; or
R⁴⁻¹ and R⁴⁻² form a morpholinyl,
R⁵ and R⁶ are independently selected from hydrogen, fluoro and chloro;
R⁷ is selected from hydrogen, fluoro and chloro;
and pharmaceutically acceptable salts thereof,
   with the proviso that the compound is not 1(3-chlorophenyl)-4-(dimethylamino)-1,8-naphthyridin-2(1H)-one or 4-amino-1-phenyl-1,8-naphthyridin-2(1H)-one.

In another embodiment, the present invention relates to compounds of the formula (I-a) 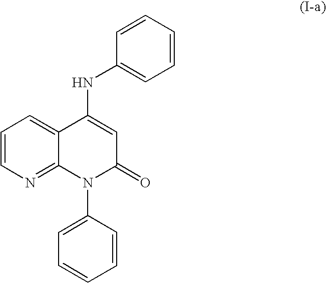
which can be substituted as described above.

In another embodiment, the present invention relates to compounds of the formula (I-b) 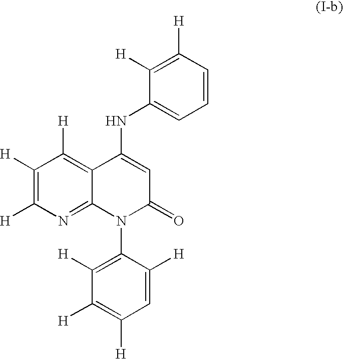
which can be substituted as described above.

Methods of the invention provide for the treatment or prevention of diabetes, including Type 1 and Type 2 diabetes, and related disorders by administration of a compound of the invention. Related disorders include maturity-onset diabetes of the young (MODY), latent autoimmune diabetes adult (LADA), impaired glucose tolerance (IGT), impaired fasting glucose (IFG), gestational diabetes, and metabolic syndrome X.

In other embodiments, methods of the invention provide for the administration of a compound of the invention in combination with a PPAR agonist, an insulin sensitizer, a sulfonylurea, an insulin secretagogue, a hepatic glucose output lowering compound, an α-glucosidase inhibitor or insulin. PPAR agonist includes rosiglitazone and pioglitazone. Sulfonylureas include glibenclamide, glimepiride, chlorpropamide, and glipizide. Insulin secretagogues include GLP-1, GIP, PACAP/VPAC receptor agonists, secretin, nateglinide, meglitinide, repaglinide, glibenclamide, glimepiride, chlorpropamide, and glipizide. α-glucosidase inhibitors include acarbose, miglitol and voglibose. A hepatic glucose output lowering compound is metformin.

In another embodiment, methods of the invention provide for the administration of a compound of the invention in combination with an HMG-CoA reductase inhibitor, nicotinic acid, a bile acid sequestrant, a fibric acid derivative, antihypertensive drug, or an anti-obesity drug. Anti-obesity drugs include a β-3 agonist, a CB-1 antagonist, and a lipase inhibitor.

In another embodiment of the invention, methods are provided for the treatment or prevention of secondary causes of diabetes, such as glucocorticoid excess, growth hormone excess, pheochromocytoma, and drug-induced diabetes.

Finally, methods of the invention provide for increasing the sensitivity of pancreatic β-cells to an insulin secretagogue, by administering a compound of the invention. Insulin secretagogues include GLP-1, GIP, PAC/VPAC receptor agonists, secretin, nateglinide, meglitinide, repaglinide, glibenclamide, glimepiride, chlorpropamide, and glipizide.

The present invention therefore provides compounds and methods for the treatment of diabetes and related disorders. These and other aspects of the invention will be more apparent from the following description.

In other words, the present invention relates to a compound of formula (I) for the treatment and/or prophylaxis of disorders, a medicament containing at least one compound of formula (I) in combination with at least one pharmaceutically acceptable, pharmaceutically safe carrier or excipient, the use of a compound of formula (I) for manufacturing a medicament for the treatment and/or prophylaxis of diabetes, a medicament containing a compound of formula (I) for the treatment and/or prophylaxis of diabetes, and a process for controlling diabetes in humans and animals by administration of an amount effective on stimulating insulin release of at least one compound of formula (I).

### DETAILED DESCRIPTION OF THE INVENTION

The invention relates generally to compounds as described in the tables and preparative examples below. Such compounds may be used in the treatment of diabetes and related disorders.

In another embodiment, the invention relates to methods of treating diabetes and related disorders by administration of compounds of the invention. Preferred methods relate to the treatment of Type 2 diabetes. In methods of the invention, compounds of the invention may be administered in combination with PPAR agonist, insulin sensitizers, sulfonylureas, insulin secretagogues, metformin, a-glucosidase inhibitors and insulin. In another embodiment, compounds of the invention are administered in combination with an HMG-CoA reductase inhibitor, nicotinic acid, a bile acid sequestrant, a fibric acid derivative, an anti-hypertensive drug or an anti-obesity drug.

In other methods of the invention, compounds of the invention are administered to treat or prevent secondary causes of diabetes or to increase the sensitivity of pancreatic β-cells to an insulin secretagogue.

Alternative Forms of Novel Compounds

Also included in the compounds of the present invention are (a) the stereoisomers thereof, (b) the pharmaceutically-acceptable salts thereof, (c) the tautomers thereof, (d) the protected acids and the conjugate acids thereof, (e) the prodrugs thereof, and (f) the solvates thereof or solvates of the salts.

(a) The Stereoisomers

The stereoisomers of these compounds may include, but are not limited to, enantiomers, diastereomers, racemic mixtures and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the inhibitors described above.

(b) The Pharmaceutically-Acceptable Salts

Pharmaceutically-acceptable salts of the compounds of the present invention include salts commonly used to form alkali metal salts or form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, heterocyclic, carboxylic and sulfonic classes of organic acids. Examples of organic and sulfonic classes of organic acids includes, but are not limited to, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicyclic, galactaric and galacturonic acid and combinations thereof.

(c) The Tautomers

Tautomers of the compounds of the invention are encompassed by the present invention. Thus, for example, a carbonyl includes its hydroxy tautomer.

(d) The Protected Acids and the Conjugate Acids

The protected acids include, but are not limited to, esters, hydroxyamino derivatives, amides and sulfonamides.

(e) The Prodrugs

The present invention includes the prodrugs and salts of the prodrugs. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, 1995) which is hereby incorporated by reference). Commonly used prodrugs are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, 1996), which is hereby incorporated by reference).

(f) The Solvates

The present invention includes the solvates and the solvates of the salts. Solvates for the purposes of the invention are those forms of the compounds that coordinate with solvent molecules to form a complex in the solid or liquid state. Hydrates are a specific form of solvates, where the coordination is with water. These include, but are not limited to, monohydrates and semihydrates.

A comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

For purposes of this invention, the chemical elements are identified in accordance with the Periodic Table of the Elements, CAS version, Handbook of Chemistry and Physics, 67th Ed., 1986-87, inside cover.

General Preparative Methods

In general, the compounds used in this invention may be prepared by standard techniques known in the art, by known processes analogous thereto, and/or by the processes described herein, using starting materials which are either commercially available or producible according to routine, conventional chemical methods. The following preparative methods are presented to aid the reader in the synthesis of the compounds of the present invention. If necessary, active groups present in the substrate will be protected against reaction with reagents or reaction under the reaction conditions. This is done according to standard methods, such as in Theodore W. Greene, Peter G. M. Wuts, Protective Groups in Organic Synthesis, 3^{rd} Ed., Wiley-Interscience, NY, N.Y., 1999.

In general, compounds of Formula (I) may be prepared from the appropriately substituted nicotinic acid through several routes summarized in Schemes I to V.

Scheme I

The nicotinic acids used in Scheme I could be purchased from commercial sources, or prepared according to literature in this field (Biorg. Med. Chem. Lett. 475-477, 2001; J. Prakt. Chem. 33, 2002; Eur. J. Org. Chem. 1371, 2001; J. Org. Chem. 6: 4618, 2000; J. Med. Chem. 40:2674, 1997; Bioorg. Med. Chem. Lett. 10:1151, 2000; U.S. Pat. No.3,838,156, etc.). 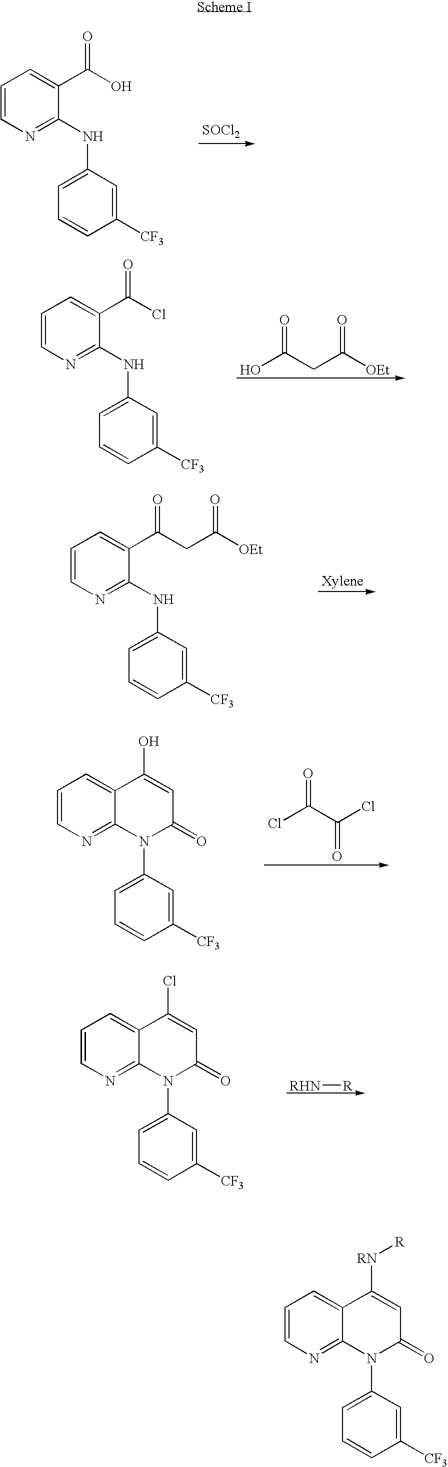
Scheme II

Scheme II illustrates manipulations of R⁶ in formula (I). These manipulations could also be applied to R⁵ and R⁷ in formula (I). 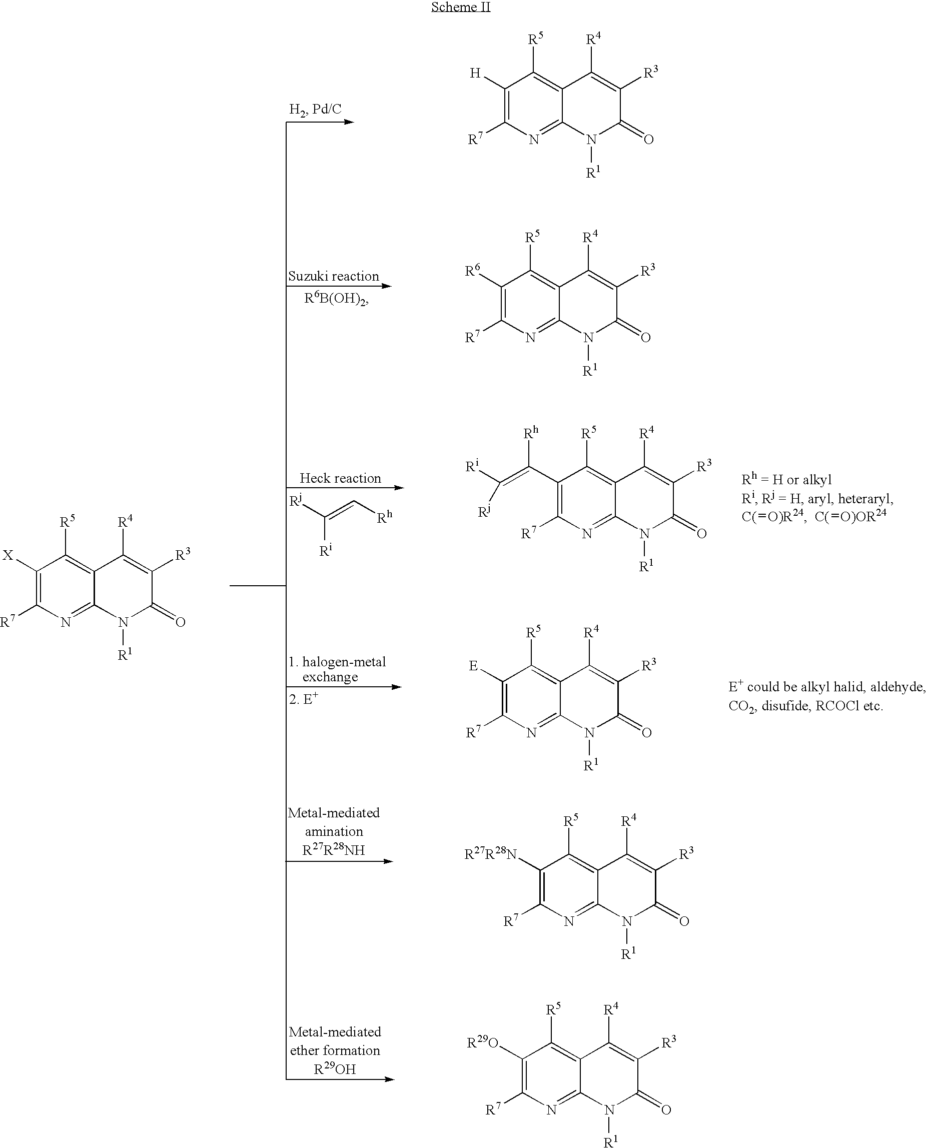
Scheme III

Scheme III illustrates manipulations on R⁷ of formula (I). These manipulations could also be applied to R⁵ in formula (I). 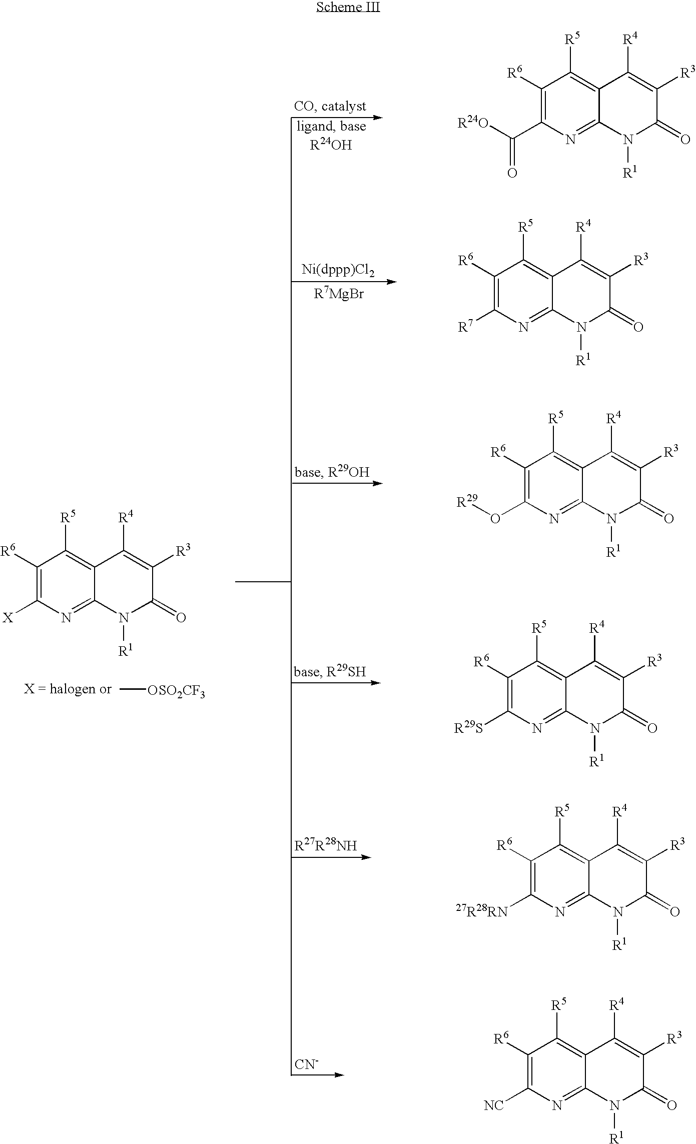
Scheme IV

Scheme IV illustrates manipulations on R⁵ of formula (I). These manipulations could also be applied to R⁷ in formula (I). 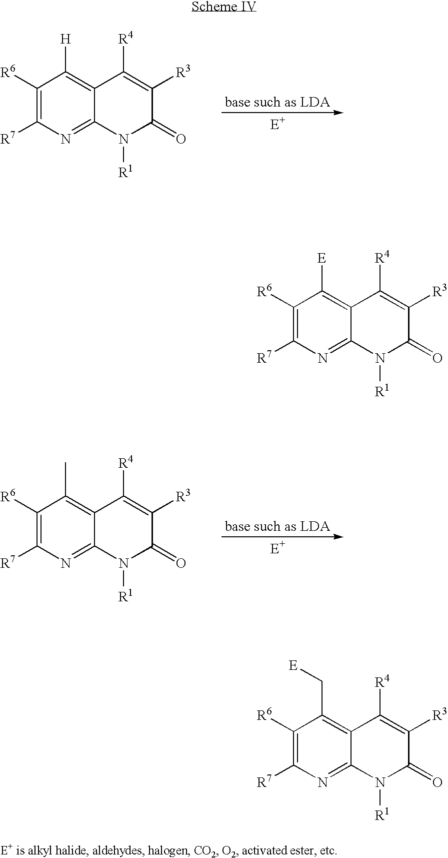
Scheme V

Scheme V illustrates the transformations of some functional groups which are present in Formula (I).

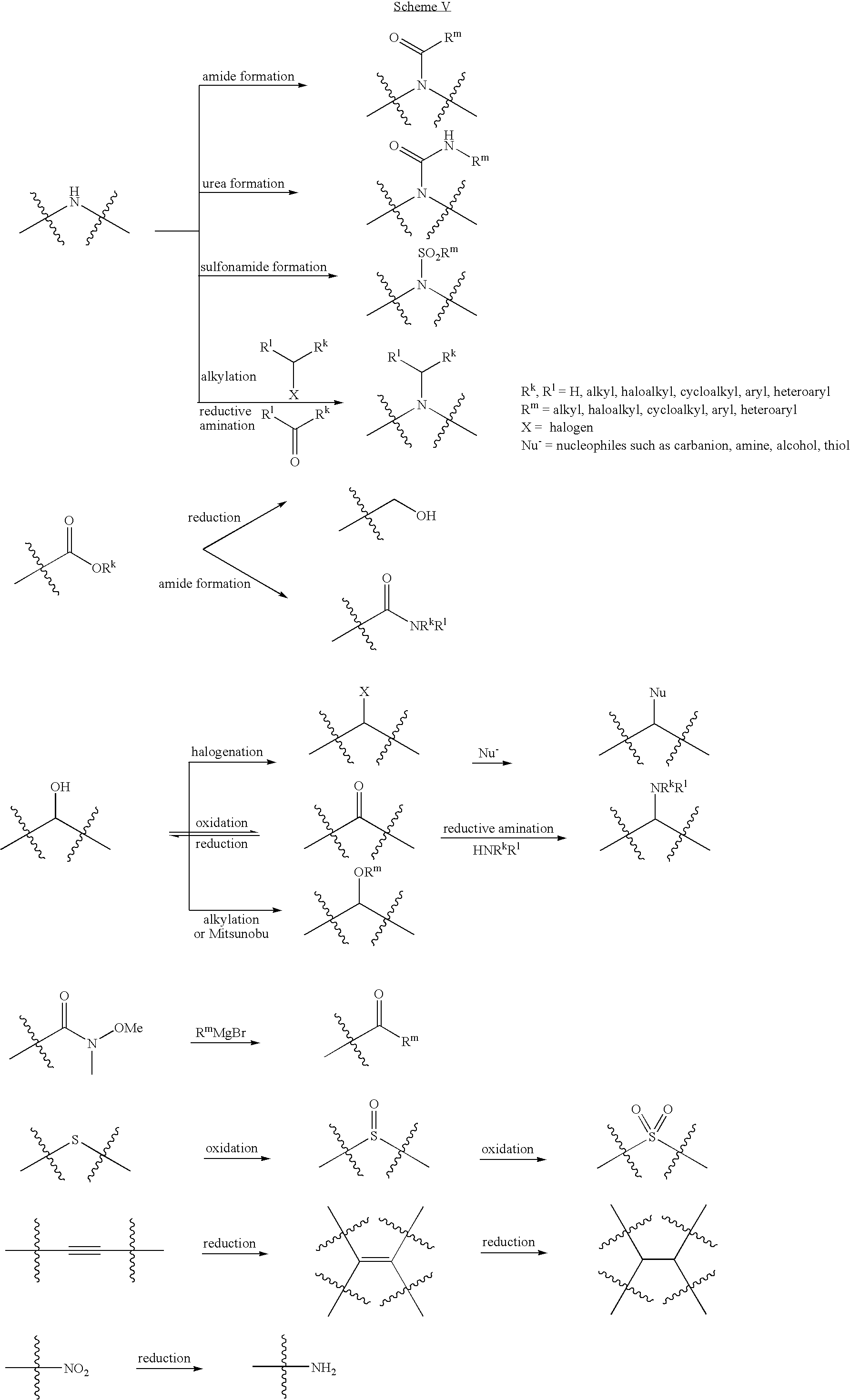

The present application thus also relates to a process for preparing compounds of the present invention, wherein compounds of formula (I), 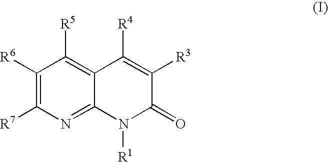
wherein R⁴ represents a leaving group, such as halogen, tosylate, mesylate or triflate, preferably chlorine,
is reacted with a compound of formula
   HNR⁴⁻¹R⁴⁻²
in the presence of a base, preferably lithium bis(trimethylsilyl)amide.

### SPECIFIC EXAMPLES OF THE INVENTION

The following specific examples are presented to illustrate the invention described herein, but should not be construed as limiting the scope of the invention in any way.

Abbreviations and Acronyms

When the following abbreviations are used throughout the disclosure, they have the following meaning:
CH₂Cl₂ methylene chloride
THF tetrahydrofuran
Na₂SO₄ anhydrous sodium sulfate
DMSO dimethylsulfoxide
EtOAc ethyl acetate
Et₃N triethylamine
HCl hydrochloric acid
¹H NMR proton nuclear magnetic resonance
HPLC high performance liquid chromatography
K₂CO₃ potassium carbonate
NH₄Cl ammonium chloride
LC/MS liquid chromatography/mass spectroscopy
MeOH methanol
NaHCO₃ sodium bicarbonate
NaOH sodium hydroxide
RT retention time
h hour
min minutes
DMF N,N-dimethylformamide
BuLi butyllithium
TLC thin layer chromatography
TFA trifluoacetic acid
LiBDS lithium hexamethyldisilazide
LDA lithium diisopropylamide
SOCl₂ thionyl chloride
AcOH acetic acid

All reactions were carried out under a positive pressure of dry argon or dry nitrogen, and were stirred magnetically unless otherwise indicated. Sensitive liquids and solutions were transferred via syringe, and introduced into reaction vessels through rubber septa. Commercial grade reagents and solvents were used without further purification.

Unless otherwise stated, the term ‘concentration under reduced pressure’ refers to use of a Buchi rotary evaporator at approximately 15 mm of Hg. All temperatures are reported uncorrected in degrees Celsius (° C.). Unless otherwise indicated, all parts and percentages are by volume.

Proton (¹H) nuclear magnetic resonance (NMR) spectra were measured with a Varian Mercury (300 MHz) or a Bruker Avance (500 MHz) spectrometer with either Me₄Si (δ 0.00) or residual protonated solvent (CHCl₃ δ 7.26; MeOH δ 3.30; DMSO δ 2.49) as standard. The NMR data of the synthesized examples, which are not disclosed in the following detailed charaterizations, are in agreements with their corresponding structural assignments.

The HPLC-MS spectra were obtained using a Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% CH₃CN in water with 0.02% TFA and B: 2% water in CH₃CN with 0.018% TFA. Gradient elution from 10% B to 95% over 3.5 minutes at a flow rate of 1.0 mL/min was used with an initial hold of 0.5 minutes and a final hold at 95% B of 0.5 minutes. Total run time was 6.5 minutes.

The IUPAC Name was obtained using the ACD/ILab Web service.

### PREPARATIVE EXAMPLES

Intermediate 1: 2-{[3-(Trifluoromethyl)phenyl]amino}nicotinoyl chloride

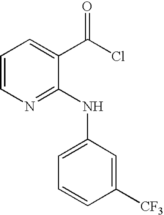

A solution of 2-[3-(trifluoromethyl)phenyl]amino]nicotinic acid (Niflumic acid, 10 g, 35.43 mmol) in thionyl chloride (31 ml, 12 eq.) was heated at reflux for 2 h. Excess thionyl chloride was evaporated and the residue was dissolved in dichloromethane and filtered through a Silica Gel plug. The filtrates were concentrated to afford the desired product (7.6 g, 71%) as a yellow solid: R_{f}=0.67 (6:1 Hexane:EtOAc).

Intermediate 2: Ethyl 3-oxo-3-(2-{[3-(trifluoromethyl)phenyl]amino}-3-pyridinyl)propanoate

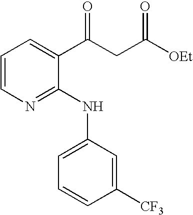

To a solution of ethyl hydrogen manolate (4.6 ml, 39.0 mmol) in THF (190 ml), cooled at −78° C., was added a solution of n-BuLi in hexane (2.5M, 32 ml, 78.38 mmol) slowly. The reaction mixture became a white milky solution. To above reaction mixture at −78° C. was added a solution of Intermediate 1 (7.6 g, 32.66 mmol) in THF (10 ml) slowly. It became an orange color solution, then changed to yellow solution. The cooling bath was removed after addition and the resulting reaction mixture was stirred at room temperature for 2 h. It was quenched with sat. NH₄Cl, extracted with ethyl acetate. The organic phase was dried over anhydrous Na₂SO₄, filtered, and concentrated to afford a brownish oil, which was purified by flash column (Silica Gel, Hexane:EtOAc=6:1 to 4:1 to 2:1). The desired product (3.52 g, 38%) was obtained as yellow oil, which became a yellow solid upon sitting. LCMS RT: 3.62 min, MH⁺=353.1, R_{f}=0.14 (6:1 Hexane:EtOAc).

Intermediate 3: 4-Hydroxy-1-[3-(trifluoromethyl)phenyl]-1,8-naphthyridin-2(1H)-one

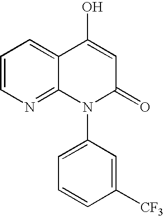

A solution of Intermediate 2 (3.52 g, 9.99 mmol) in xylene (50 ml) was heated at 130° C. overnight (18 h). It became a suspension upon heating. The reaction mixture was cooled to room temperature and the precipitated desired product was collected by filtration as a white solid. It was washed with ethyl acetate. The filtrates were combined, concentrated, dissolved in Xylene, and heated at 130° C. overnight again. The desired product was collected by filtration again. Totally, 2 g of desired product (65%) was obtained: LCMS RT=2.45 min, MH⁺=307.2.

Intermediate 4: 4-Chloro-1-[3-(trifluoromethyl)phenyl]-1,8-naphthyridin-2(1H)-one

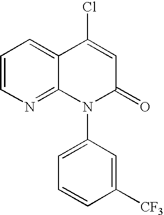

Intermediate 3 (1 g, 3.27 mmol) was dissolved in mixture of oxalyl chloride (13 ml) and a solution of 2M oxalyl chloride in dichloromethane (13 ml). The reaction mixture was stirred at room temperature overnight (18 h). Solvent and excess oxalyl chloride were evaporated, the residue was suspended in a small amount of ethyl acetate, and filtered to get rid off unreacted starting material. The filtrates were concentrated and purified by flash column (Silica Gel, Hexane:EtOAc=3:1). The desired product (633 mg, 66%) was obtained as a white solid: LCMS RT=2.74, MH⁺=325.4, R_{f}=0.10 (6:1 Hexane:EtOAc).

Final Products:

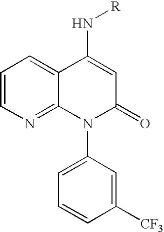

A reaction mixture of intermediate 4 (30 mg, 0.0924 mmol, 1 eq.), amine (2 eq.), and 1M Lithium bis(trimethylsilyl)amide in hexane (LiHMDS, 2 eq.) in 1,4-dioxane was stirred either at room temperature or at reflux overnight (18 h). It was quenched with sat. NH₄Cl, extracted with dichloromethane. The organic phase was dried over anhydrous Na₂SO₄, filtered, concentrated and purified by flash column (Silica Gel, Hexane:EtOAc=2:1). The desired products were obtained in the yield of 18% to 60%.

Compounds that may be synthesized via the above-described procedures include:

TABLE 1

| | | LCMS, | |
|---|---|---|---|
| Example | | RT | |
| No. | Structure | (min) | [MH+] |
| 1 | 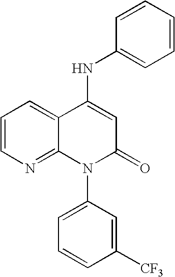 | 2.90 | 382.3 |
| 2 | 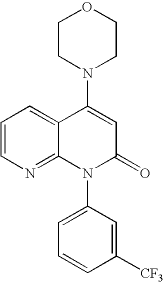 | 2.68 | 376.3 |
| 3 | 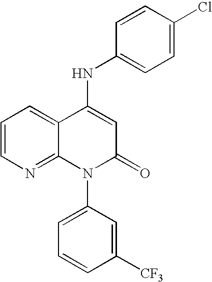 | 3.15 | 416.5 |
| 4 | 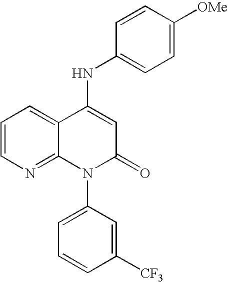 | 3.24 | 412.4 |
| 5 | 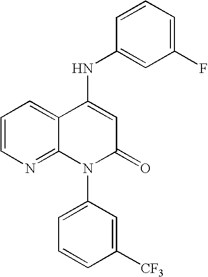 | 3.01 | 400.4 |
| 6 | 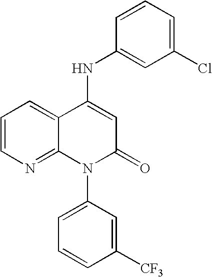 | 3.25 | 416.2 |
| 7 | 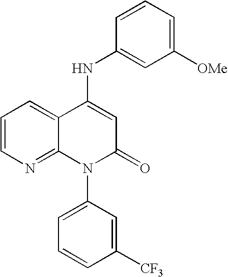 | 3.05 | 412.3 |
| 8 | 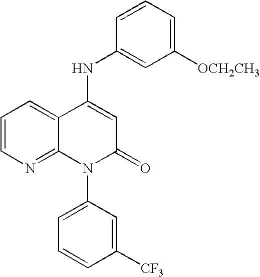 | 3.21 | 426.3 |
| 9 | 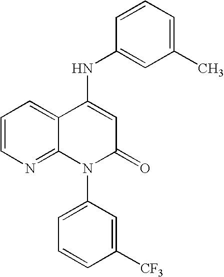 | 3.20 | 396.3 |
| 10 | 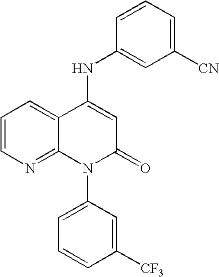 | 2.88 | 407.6 |
| 11 | 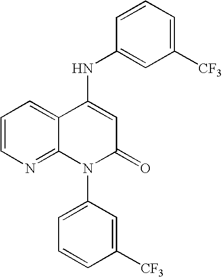 | 3.36 | 450.2 |
| 12 | 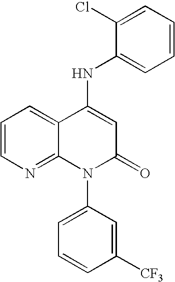 | 3.05 | 416.5 |

The compounds of the present invention may be employed in the treatment of diabetes, including both type 1 and type 2 diabetes (non-insulin dependent diabetes mellitus). Such treatment may also delay the onset of diabetes and diabetic complications. The compounds may be used to prevent subjects with impaired glucose tolerance from proceeding to develop type 2 diabetes. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); and metabolic syndrome X.

The compounds of the present invention may also be effective in such disorders as obesity, and in the treatment of atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease.

The compounds of the present invention may also be useful for treating physiological disorders related to, for example, cell differentiation to produce lipid accumulating cells, regulation of insulin sensitivity and blood glucose levels, which are involved in, for example, abnormal pancreatic beta-cell function, insulin secreting tumors and/or autoimmune hypoglycemia due to autoantibodies to insulin, autoantibodies to the insulin receptor, or autoantibodies that are stimulatory to pancreatic beta-cells, macrophage differentiation which leads to the formation of atherosclerotic plaques, inflammatory response, carcinogenesis, hyperplasia, adipocyte gene expression, adipocyte differentiation, reduction in the pancreatic beta-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

Compounds of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

The compounds of the present invention may be used alone or in combination with additional therapies and/or compounds known to those skilled in the art in the treatment of diabetes and related disorders. Alternatively, the methods and compounds described herein may be used, partially or completely, in combination therapy.

The compounds of the invention may also be administered in combination with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, insulin and anti-obesity drugs. Such therapies may be administered prior to, concurrently with or following administration of the compounds of the invention. Insulin includes both long and short acting forms and formulations of insulin. PPAR agonist may include agonists of any of the PPAR subunits or combinations thereof. For example, PPAR agonist may include agonists of PPAR-α, PPAR-γ, PPAR-δ or any combination of two or three of the subunits of PPAR. PPAR agonists include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like); biguanides such as metformin and phenformin; protein tyrosine phosphatase-1B (PTP-1B) inhibitors; dipeptidyl peptidase TV (DP-IV) inhibitors; and thiazolidinediones and non-thiazolidinediones. Hepatic glucose output lowering compounds that may be useful in treating diabetes when administered with a compound of the invention include metformnin, such as Glucophage and Glucophage XR. Insulin secretagogues that may be useful in treating diabetes when administered with a compound of the invention include sulfonylurea and non-sulfonylurea drugs: GLP-1, GIP, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin. In one embodiment of the invention, compounds of the invention are used in combination with insulin secretagogues to increase the sensitivity of pancreatic β-cells to the insulin secretagogue.

Compounds of the invention may also be used in methods of the invention in combination with anti-obesity drugs. Anti-obesity drugs include β-3 agonists; CB-1 antagonists; neuropeptide Y5 inhibitors; appetite suppressants, such as, for example, sibutramine (Meridia); and lipase inhibitors, such as, for example, orlistat (Xenical).

Compounds of the invention may also be used in methods of the invention in combination with drugs commonly used to treat lipid disorders in diabetic patients. Such drugs include, but are not limited to, HMG-CoA reductase inhibitors, nicotinic acid, lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention may also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a compound of the invention in combination with an insulin sensitizer and an anti-obesity drug). Such co-therapies may be administered in the form of pharmaceutical compositions, as described above.

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a diabetic condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

Based on well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and trietbanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

It should be apparent to one of ordinary skill in the art that changes and modifications can be made to this invention without departing from the spirit or scope of the invention as it is set forth herein.

### Biological Evaluation

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro, ex vivo, and in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia, the following assays may be used.

### Example 1

### Preparation of Pseudo Islets in 96-Well Plates

Pancreata from four Sprague Dawley rats were divided into small pieces approximately 1 mm² or smaller in size. The tissue was then rinsed three times with Hanks-Hepes buffer (127 mM NaCl, 5.4 mM KCl, 0.34 mM Na₂HPO₄, 4.4 mM KH₂PO₄, 20 mM HEPES (4-(2-Hydroxyethyl)piperazine-1-ethanesulfonic acid), 1.2 mM CaCl₂/5 mM glucose), and digested with collagenase (Liberase, 0.25 mg/ml, Roche Diagnostic Corp., Indianapolis, Ind., USA) at 37° C. in a water bath shaker for 10 minutes.

The digested pancreata tissue was rinsed three times with 50 ml of Hanks-Hepes buffer to remove the collagenase. The tissue pellet was then filtered through a 250 μm filter and the filtrate was mixed with 16 ml of 27% Ficoll (Sigma, St. Louis, Mo., USA) w/v in Hanks-Hepes buffer. Three layers of Ficoll (23%, 20.5%, and 11%, respectively; 8 ml of each concentration) were then loaded on top of the mixture of islet tissue in 27% Ficoll to form a gradient.

The Ficoll gradient was then centrifuged at 1,600 rpm for 10 minutes at room temperature. The pancreatic islets were concentrated at the interphase between 11% and 20.5%, and between 20.5% and 23% depending on the size of islets. The islets were collected from the two interphases and rinsed twice with Ca⁺⁺-free Hanks-Hepes buffer. The islets were then suspended in 5 ml Ca⁺⁺-free Hanks-Hepes buffer containing 1 mM EDTA and incubated for 8 minutes at room temperature.

Trypsin and DNAse I were added to the islet suspension for a final concentration of 25 μg/ml and 2 μg/ml, respectively. This suspension was incubated with shaking at 30° C. for 10 minutes. The trypsin digestion was stopped by adding 40 ml RPMI 1640 (GIBCO Life Technologies, Invitrogen, Carlsbad, Calif.) with 10% FBS. The trypsin digested islet cells were then filtered through a 63 μm nylon filter (PGC Scientific, Frederick, Md.) to remove large cell clusters.

The dispersed islet cells were then washed, counted using hemacytometer under the microscope, and seeded into “V-bottom” 96-well plates (2,500 cells per well). The dispersed islet cell suspension was then centrifuged at 1,000 rpm for 5 minutes. The Hanks-Hepes buffer was removed and replaced with 200 μl RPMI 1640 medium containing 10% FBS, 1% Penicillin—Streptomycin, and 2 mM L-glutamine. Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% CO₂, and then used for assays.

### Example 2

### Pseudo Islet Incubation with 3T3-L1 Cells

Dispersed islet cells (prepared by the method described in Example 1) were washed with regular RPMI 1640 medium with 10% FBS, counted using hemacytometer under the microscope, and seeded into “V-bottom” 96-well plates with 3T3-L1 cells (2,500 islet cells and 1,250 3T3-L1 cells per well). The cell suspension was then centrifuged at 1,000 rpm for 5 minutes to collect the dispersed islet cells concentrated at the V-bottom of the plate forming pseudo islets. These pseudo islets were then co-cultured with the 3T3-L1 cells overnight in a cell culture incubator at 37° C. with 5% CO₂, and then used for assays.

### Example 3

### Freezing and Thawing of Pseudo Islets

Dispersed islet cells (prepared by the method described in Example 1) were counted as described above and diluted in regular RPMI 1640 medium with 10% FBS and 10% DMSO to a concentration of 2×10⁵ cells per ml. An aliquot (1 ml) was transferred to a cryotube and the cryotube was placed in a rack in the vapor phase in a liquid nitrogen tank prior to freezing in liquid nitrogen.

Cells were thawed and then washed with regular medium and seeded into “V-bottom” 96-well plates (5,000 cells per well). Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% CO₂, and then used for assays.

### Example 4

### Static Pseudo Islet Incubation for Insulin Release Assay

Pseudo islets were prepared by the method described in Example 1. Following an overnight incubation, the RPMI 1640 medium was removed and replaced by 100 μL Krebs-Ringer-Hepes buffer (115 mM NaCl, 5.0 mM KCl, 24 mM NaHCO₃, 2.2 mM CaCl₂, 1 mM MgCl₂, 20 mM HEPES, 0.25% BSA (Bovine serum albumin), 0.002% Phenol Red, pH 7.35-7.40). The cell suspension was then centrifuged for 5 minutes at 1,000 rpm to pellet the dispersed islet cells.

Pseudo islets in 96-well plates were incubated in a water bath at 37° C. continuously gassed with 95% O₂/5% CO₂ for pre-incubation for 30 minutes. The pre-incubation buffer was removed and replaced with 50 μl incubation buffer (Krebs-Ringer-Hepes buffer, pH 7.35-7.40) containing various test substrates.

The 96-well plate was centrifuged again at 1,000 rpm for 5 minutes to form pseudo islets. These pseudo islets in 96-well plates were statically incubated in a water bath at 37° C. continuously gassed with 95% O₂/5% CO₂ for 60 minutes. The incubation buffer (25 μl) was collected after the 60-minute incubation and used for an insulin content assay (ELISA assay, ALPCO, N.H., USA).

### Example 5

### Static Pseudo Islet Incubation for Insulin Biosynthesis

Pseudo islets are prepared as described in Example 1. After an overnight culture, the pseudo islets are preincubated in KRBH (Krebs-Ringer-Hepes buffer, 135 mM NaCl, 3.6 mM KCl, 10 mM HEPES, 5 mM NaHCO₃, 0.5 mM NaH₂PO₄, 0.5 mM MgCl₂, 1.5 mM CaCl₂, 0.1% Bovine Serum Albumin) containing 3 mM glucose for 30 minutes at 37° C., and then incubated for 90 minutes at 37° C. with test compounds and 2 μM ³H-Leucine (100 μL) (Amersham, Piscataway, N.J., USA). The pseudo islets are then washed 3× with KRBH containing 1 mM leucine (Sigma, St. Louis, Mo., USA), lysed in 2 mM acetic acid (100 μl), sonicated for 15 seconds, and neutralized with 10 N NaOH (20 μl). HEPES (50 mM) containing 0.1% Triton X-100 (Calbiochem, San Diego, Calif., USA) is added to bring the volume to 1 ml and the samples are spun for 10 minutes at 1750×g. Protein A Agarose (50 μl per sample) is preincubated with anti-insulin antibody (Linco, St. Charles, Mo., USA) (100 μl per sample) for 2 hours and washed twice. The antibody bead mixture (50 μl) was added to 750 μl of sample and incubated overnight at 4° C. The immunoprecipitates are washed 3× with HEPES (50 mM) containing 0.1% Triton X-100. The beads are then counted in a scintillation counter.

### Example 6

### Static Pseudo Islet Incubation for Glucagon Release

Pseudo islets are prepared as described in Example 1. Following an overnight incubation, the RPMI 1640 medium was removed and replaced by 100 μl Krebs-Ringer-Hepes buffer (115 mM NaCl, 5.0 mM KCl, 24 mM NaHCO₃, 2.2 mM CaCl₂, 1 mM MgCl₂, 20 mM HEPES, 0.25% BSA, 0.002% Phenol Red, pH 7.35-7.40). The cell suspension was then centrifuged for 5 minutes at 1,000 rpm to pellet the dispersed islet cells.

Pseudo islets in 96-well plates were incubated in a water bath at 37° C. continuously gassed with 95% O₂/5% CO₂ for pre-incubation for 30 minutes. The pre-incubation buffer was removed and replaced with 50 μl incubation buffer (Krebs-Ringer-Hepes buffer, pH 7.35-7.40) containing various test compounds.

The 96-well plate was centrifuged again at 1,000 rpm for 5 minutes to form pseudo islets. These pseudo islets in 96-well plates were statically incubated in a water bath at 37° C. continuously gassed with 95% O₂/5% CO₂ for 60 minutes. The incubation buffer (25 μl) was collected after the 60-minute incubation and used for a glucagon content assay (Glucagon RIA kit; Linco, St. Charles, Mo., USA).

### Example 7

### Assay for Identifying Insulinotropic Compounds

Pseudo islets were prepared as described in Example 1. The dispersed islet cells were then washed, counted using a hemacytometer, and seeded into “V-bottom” 96-well plates (2,500 cells per well) with 200 μl RPMI 1640 medium containing 10% FBS, 1% Penicillin—Streptomycin, and 2 mM L-glutamine. Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% CO₂.

Following the overnight incubation, the RPMI 1640 medium was removed and replaced by 100 μl Krebs-Ringer-HEPES buffer (115 mM NaCl, 5.0 mM KCl, 24 mM NaHCO₃, 2.2 mM CaCl₂, 1 mM MgCl₂, 20 mM HEPES, 0.25% BSA, 0.002% Phenol Red, pH 7.35-7.40) with 3 mM glucose. The cell suspension was then centrifuged for 5 minutes at 1,000 rpm to pellet the dispersed islet cells.

The pseudo islets in 96-well plates were incubated in a water bath at 37° C. continuously gassed with 95% O₂/5% CO₂ for a pre-incubation of 30 minutes. The pre-incubation buffer was removed and replaced with 50 μl incubation buffer (Krebs-Ringer-HEPES buffer, pH 7.35-7.40) containing the test compounds. The 96-well plates were centrifuged again at 1,000 rpm for 5 minutes to form pseudo islets. These pseudo islets were then statically incubated in a water bath at 37° C. continuously gassed with 95% O₂/5% CO₂ for 30 minutes. The incubation buffer (25 μl) was collected after the 30-minute incubation and used for an insulin content assay.

Compounds of the present invention stimulate insulin release over basal levels. For example, compounds 2, 6-10 and 12 were found to stimulate insulin release about 1.3- to 2-fold over basal insulin release.

Pharmaceutical compositions

The compounds according to the invention can be converted into pharmaceutical preparations as follows:

Tablet:

Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate. Tablet weight: 212 mg, diameter 8 mm, curvature radius 12 mm.

Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

Orally Administrable Suspension:

Composition:

1000 mg of the compound of Example 1, 1000 mg of ethanol (96%), 400 mg of Rhodigel (xanthan gum from FMC, Pennsylvania, USA) and 99 g of water.

A single dose of 100 mg of the compound according to the invention is provided by 10 ml of oral suspension.

Preparation:

The Rhodigel is suspended in ethanol and the active component is added to the suspension. The water is added with stirring. Stirring is continued for about 6 h until the swelling of the Rhodigel is complete.

What is claimed:

1. A compound of the formula 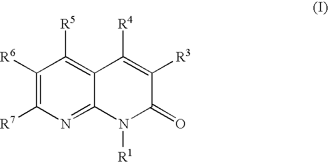
wherein
R¹ is selected from alkyl of 1-8 carbon atoms, alkenyl of 2-8 carbon atoms, alkynyl of 2-8 carbon atoms, and A-R⁹,
or
R¹ is selected from aryl of 6-10 carbon atoms, heteroaryl of 2-9 carbon atoms and 1-4 heteroatoms selected from N, S(═O)₀₋₂ and O, cycloalkyl of 3-8 carbon atoms, cycloalkenyl of 4-8 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroa selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), all of which may be substituted with 1-3 of R¹⁰;
R¹⁰ is selected from nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms, haloalkoxy of 1-6 carbon atoms, cycloalkoxy of 3-6 carbon atoms, aryl of 6-10 carbon atoms, heteroaryl of 2-9 carbon atoms and 1-4 heteroatoms selected from N, S(═O)₀₋₂ and O, NR¹¹R¹², C(═O)OR¹¹, C(═O)NHR¹¹, NHC(═O)R¹³, NHS(═O)₂R¹³, S(═O)₀₋₂R¹³, S(═O)₂NHR¹¹, cycloalkyl of 3-6 carbon atoms, cycloalkenyl of 3-6 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O);
R¹³ is selected from alkyl of 1-6 carbon atoms, alkenyl of 2-6 carbon atoms, alkynyl of 2-6 carbon atoms, haloalkyl of 1-6 carbon atoms, cycloalkyl of 3-6 carbon atoms, and cycloalkenyl of 4-6 carbon atoms;
R¹¹ and R¹² are independently selected from hydrogen, alkyl of 1-6 carbon atoms, alkenyl of 2-6 carbon atoms, alkynyl of 2-6 carbon atoms, haloalkyl of 1-6 carbon atoms, cycloalkyl of 3-6 carbon atoms, and cycloalkenyl of 4-6 carbon atoms;
A is selected from alkyl of 1-8 carbon atoms, alkenyl of 2-8 carbon atoms, alkynyl of 2-8 carbon atoms, and haloalkyl of 1-8 carbon atoms;
R⁹ is selected from hydroxy, alkoxy of 1-6 carbon atoms, cycloalkoxy of 3-6 carbon atoms, O-A-R¹⁴, NR¹¹R¹²; or
R⁹ is selected from aryl of 6-10 carbon atoms, heteroaryl of 2-9 carbon atoms and 1-4 heteroatoms selected from N, S(═O)₀₋₂ and O, cylcoalkyl of 3-8 carbon atoms, cycloalkenyl of 5-8 carbon atoms, all of which may be substituted with 1-3 of R¹⁰, or
R⁹ is selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R¹⁴ is selected from cylcoalkyl of 3-8 carbon atoms, cycloalkenyl of 5-8 carbon atoms, 5-7 membered heterocycloalkyl of 3-6 carbon atoms and 1-2 heteroatoms selected from N, S(═O)₀₋₂ and O, and 5-7 membered heterocycloalkenyl of 3-6 carbon atoms and 1-2 heteroatoms selected from N, S(═O)₀₋₂ and O, all of which may be substituted with 1-3 of R¹⁰;
R³ is hydrogen,
R⁴ is —NR⁴⁻¹R⁴⁻²;
R⁴⁻¹ is selected from the group consisting of hydrogen, alkyl of 1-8 carbon atoms, alkenyl of 2-8 carbon atoms, alkynyl of 2-8 carbon atoms and haloalkyl of 1-8 carbon atoms;
R⁴⁻² is selected from the group consisting of hydrogen, alkyl of 1-8 carbon atoms, alkenyl of 2-8 carbon atoms, alkynyl of 2-8 carbon atoms, haloalkyl of 1-8 carbon atoms, aryl of 6-10 carbon atoms, heteroaryl of 2-9 carbon atoms and 1-4 heteroatoms, 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said aryl, hereroaryl, heterocycloalkyl or heterocycloalkenyl may be substituted with one to three substituents selected from the group consisting of nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms and haloalkoxy of 1-6 carbon atoms, or
R⁴⁻¹ and R⁴⁻² form a 5-7 membered heterocycloalkyl of 3-6 carbon atoms and 1-2 heteroatoms selected from N, S(═)₀₋₂ and O, wherein said heterocycloalkyl may be substituted with one to three substituents selected from the group consisting of nitro, nitrile, hydroxy, halogen, acyl of 1-6 carbon atoms, alkyl of 1-6 carbon atoms, alkenyl of 2-6 carbon atoms, alkynyl of 2-6 carbon atoms, haloalkyl of 1-6 carbon atoms, alkoxy of 1-6 carbon atoms and haloalkoxy of 1-6 carbon atoms,
R⁵ and R⁶ are independently selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms, all of which may be substituted with 1-3 of R¹⁰,
or
R⁵ and R⁶ are independently selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, A-R²³, A-NR²⁴R²⁵, C(═O)R²⁴, C(═O)OR²⁴, C(═O)NR²⁴R²⁵, S(═O)₂R²⁶, A-C(═O)R²⁴, A-C(═O)OR²⁴, or A-C(═O)NR²⁴R²⁵,
or
R⁵ and R⁶ are independently selected from hydrogen, halogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and AC(═O)NR²⁴R²⁵;
Q is selected from 0 and S(═O)₀₋₂;
R²² is selected from hydrogen, alkyl of 1-8 carbon atoms, haloalkyl of 1-8 carbon atoms, and cycloalkyl of 3-8 carbon atoms;
R²³ is selected from hydroxy, alkoxy of 1-8 carbon atoms, haloalkoxy of 1-8 carbon atoms, and cycloalkoxy of 3-8 carbon atoms, or
R²³ is selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
R²³ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
with the proviso for A(OR²²)—R²³ that when R²³ is selected from hydroxy, alkoxy of 1-8 carbon atoms, haloalkoxy of 1-8 carbon atoms, and cycloalkoxy of 3-8 carbon atoms, A is not CH;
R²⁴ and R²⁵ are independently selected from hydrogen, alkyl of 1-6 carbon atoms, alkenyl of 2-6 carbon atoms, alkynyl of 2-6 carbon atoms, haloalkyl of 1-6 carbon atoms, and A-R²³, or
R²⁴ and R²⁵ are independently selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
R²⁴ and R²⁵ are independently selected from 5-7 membered heterocycloalkyl of 3-6 carbon atoms and 1-2 heteroatoms selected from N, O, S(═)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, or
R²⁴ and R²⁵ combine, together with the nitrogen atom to which they are attached, to form a 5-7 membered heterocycloalkyl of 3-6 carbon atoms and 1-2 heteroatoms selected from N, S(═O)₀₋₂, and O, a 5-7 membered heterocycloalkenyl of 3-6 carbon atoms and 1-2 heteroatoms selected from N, S(═O)₀₋₂, and O, or a heteroaryl of 2-9 carbon atoms and 1-4 heteroatoms, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), all of which may be substituted with 1-3 of R¹⁰;
R²⁶ is selected from alkyl of 1-6 carbon atoms, alkenyl of 2-6 carbon atoms, alkynyl of 2-6 carbon atoms, haloalkyl of 1-6 carbon atoms, A(OR²²)—R²³, and A-R²³, or
R²⁶ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
R²⁶ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R²⁷ is selected from hydrogen, alkyl of 1-6 carbon atoms, alkenyl of 2-6 carbon atoms, alkynyl of 2-6 carbon atoms, haloalkyl of 1-6 carbon atoms, and A-R²³, or
R²⁷ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═O)₀₋₂, and O, all of which may be substituted with 1-3 of R¹⁰, or
R²⁷ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R²⁸ is selected from hydrogen, alkyl of 1-6 carbon atoms, alkenyl of 2-6 carbon atoms, alkynyl of 2-6 carbon atoms, haloalkyl of 1-6 carbon atoms, A-R²³, C(═O)R²⁴, C(═O)OR²⁶, C(═O)NR²⁵R³⁰, S(═O)₂R²⁶, A-C(═O)R²⁴, A-C(═O)OR²⁴, and A-C(═O)NR²⁴ R²⁵, or
R²⁸ is selected from cycloalkyl of 3-6 carbon atoms, cycloalkenyl of 3-6 carbon atoms, aryl of 6-10 carbon atoms, heteroaryl of 2-9 carbon atoms and 1-4 heteroatoms selected from N, S(═O)₀₋₂, and O, all of which may be substituted with 1-3 of R¹⁰, or
R²⁸ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R³⁰ is selected from alkyl of 1-6 carbon atoms, alkenyl of 2-6 carbon atoms, alkynyl of 2-6 carbon atoms, haloalkyl of 1-6 carbon atoms, A(OR²²)—R²³, and A-R²³, or
R³⁰ is selected from cycloalkyl of 3-6 carbon atoms, cycloalkenyl of 3-6 carbon atoms, aryl of 6-10 carbon atoms, and heteroaryl of 2-9 carbon atoms and 1-4 heteroatoms selected from N, S(═)₀₋₂, and O, all of which may be substituted with 1-3 of R¹⁰, or
R³⁰ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)₀₋₂, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, or
R²⁵ and R³⁰ combine, together with the nitrogen atom to which they are attached, to form a 5-7 membered heterocycloalkyl of 3-6 carbon atoms and 1-2 heteroatoms selected from N, S(═)₀₋₂, and O, a 5-7 membered heterocycloalkenyl of 3-6 carbon atoms and 1-2 heteroatoms selected from N, S(═)₀₋₂, and O, or a heteroaryl of 2-9 carbon atoms and 1-4 heteroatoms, all of which may be substituted with 1-3 of R¹⁰;
R²⁹ is selected from alkyl of 1-6 carbon atoms, alkenyl of 2-6 carbon atoms, alkynyl of 2-6 carbon atoms, haloalkyl of 1-6 carbon atoms, A-R²³, A-C(═O)R²⁴, A-C(═O)OR²⁴, A-C(═O)NR²⁴R²⁵, A-NR²⁷R²⁸, or
R²⁹ is selected from cycloalkyl of 3-6 carbon atoms, cycloalkenyl of 3-6 carbon atoms, aryl of 6-10 carbon atoms, heteroaryl of 2-9 carbon atoms and 1-4 heteroatoms selected from N, S(═O)₀₋₂, and O, all of which may be substituted with 1-3 of R¹⁰, or
R²⁹ is selected from 5-7 membered heterocycloalkyl of 3-6 carbon atoms and 1-2 heteroatoms selected from N, O, S(═O)₀₋₂, and 5-7 membered heterocycloalkenyl of 3-6 carbon atoms and 1-2 heteroatoms selected from N, O, S(═O)0-2, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰;
R⁷ is selected from cycloalkyl of 3-8 carbon atoms, cycloalkenyl of 4-8 carbon atoms, aryl of 6-10 carbon atoms, and heteroaryl of 2-9 carbon atoms and 1-4 heteroatoms, all of which may be substituted with 1-3 of R¹⁰,
or
R⁷ is selected from 5-7 membered heterocycloalkyl of 3-6 carbon atoms and 1-2 heteroatoms selected from N, S(═O)₀₋₂ and O and 5-7 membered heterocycloalkenyl of 3-6 carbon atoms and 1-2 heteroatoms selected from N, S(═O)₀₋₂ and O, wherein said heterocycloalkyl and said heterocycloalkenyl may further be fused with phenyl or a 5-6 membered heteroaryl of 2-5 carbon atoms and 1-3 heteroatoms selected from N, S(═O)₀₋₂ and O, and/or wherein one or more of the carbon atoms in said heterocycloalkyl or heterocycloalkenyl may be oxidized to C(═O), wherein said heterocycloalkyl or said heterocycloalkenyl may be substituted with 1-3 of R¹⁰, A(OR²²)—R²³, A-R²³, A-NR²⁴R²⁵, C(═O)R²⁴, C(═O)OR²⁴, C(═O)NR²⁴R²⁵, S(═O)₂R²⁶, A-C(═O)R²⁴, A-C(═O)OR²⁴, or A-C(═O)NR²⁴R²⁵,
or
R⁷ is selected from hydrogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
and a pharmaceutically acceptable salt thereof,
with the proviso that the compound is not 1-(3-chlorophenyl)-4dimethylamino)-1,8-naphthyridin-2(1H)-one or 4-amino-1-phenyl-1,8-naphthyridin-2(1H)-one.

2. The compound of claim 1, wherein
R¹⁰ is selected from aryl of 6 or 10 carbon atoms, which may be substituted with 1-3 of R¹⁰;
R¹⁰ is selected from nitro, nitrile, hydroxy, halogen, acyl of 1-6 carbon atoms, alkyl of 1-6 carbon atoms, haloalkyl of 1-6 carbon atoms, alkoxy of 1-6 carbon atoms, haloalkoxy of 1-6 carbon atoms, cycloalkoxy of 3-6 carbon atoms, phenyl, heteroaryl selected from thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl, NR¹¹R¹², C(═O)OR¹¹, C(═O)NHR¹¹, NHC(═O)R¹³, NHS(═O)₂R¹³, S(═O)₀₋₂R¹³, S(═O)₂NHR¹¹, cyclopropyl, cyclopentyl, cyclohexyl, and heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl;
R¹³ is selected from alkyl of 1-6 carbon atoms, haloalkyl of 1-6 carbon atoms, and cycloalkyl of 3-6 carbon atoms;
R¹¹ and R¹² are independently selected from hydrogen, alkyl of 1-6 carbon atoms, haloalkyl of 1-6 carbon atoms, and cycloalkyl of 3-6 carbon atoms;
A is selected from alkyl of 1-8 carbon atoms and haloalkyl of 1-8 carbon atoms;
R³ is hydrogen,
R⁴ is —NR⁴⁻¹R⁴⁻²;
R⁴⁻¹ is selected from the group consisting of hydrogen, alkyl of 1-8 carbon atoms and haloalkyl of 1-8 carbon atoms;
R⁴⁻² is selected from the group consisting of hydrogen, alkyl of 1-8 carbon atoms, haloalkyl of 1-8 carbon atoms, aryl of 6 or 10 carbon atoms, heteroaryl selected from thienyl, furyl, pyrrolyl, thiazolyl, oxazolyl, imidazolyl, pyridyl, pyrimidyl, pyridazinyt, indolyl, indazolyl, benzofuranyl, benzothiophenyl, quinolinyl, isoquinolinyl, heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said aryl, hereroaryl or heterocycloalkyl may be substituted with one to three substituents selected from the group consisting of nitro, nitrile, hydroxy, halogen, acyl of 1-6 carbon atoms, alkyl of 1-6 carbon atoms, haloalkyl of 1-6 carbon atoms, alkoxy of 1-6 carbon atoms and haloalkoxy of 1-6 carbon atoms, or
R⁴⁻¹ and R⁴⁻² form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may be substituted with one to three substituents selected from the group consisting of nitro, nitrile, hydroxy, halogen, acyl of 1-6 carbon atoms, alkyl of 1-6 carbon atoms, haloalkyl of 1-6 carbon atoms, alkoxy of 1-6 carbon atoms and haloalkoxy of 1-6 carbon atoms,
R⁵ and R⁶ are independently selected from cycloalkyl of 3-8 carbon atoms, aryl of 6-10 carbon atoms, and heteroaryl of 2-9 carbon atoms and 1-4 heteroatoms, all of which may be substituted with 1-3 of R¹⁰,
or
R⁵ and R⁶ are heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl,
or
R⁵ and R⁶ are independently selected from hydrogen, halogen, nitrile, nitro, hydroxy, alkyl of 1-8 carbon atoms, alkenyl of 2-8 carbon atoms, alkynyl of 2-8 carbon atoms, haloalkyl of 1-8 carbon atoms, alkoxy of 1-8 carbon atoms, haloalkoxy of 1-8 carbon atoms, cycloalkoxy of 3-8 carbon atoms, A²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
Q is selected from O and S(═O)₀₋₂;
R²² is selected from hydrogen, alkyl of 1-8 carbon atoms, haloalkyl of 1-8 carbon atoms, and cycloalkyl of 3-8 carbon atoms;
R²³ is selected from hydroxy, alkoxy of 1-8 carbon atoms, haloalkoxy of 1-8 carbon atoms, and cycloalkoxy of 3-8 carbon atoms, or
R²³ is selected from cycloalkyl of 3-8 carbon atoms, aryl of 6 or 10 carbon atoms, and heteroaryl of 2-9 carbon atoms and 1-4 heteroatoms selected from N, S(═O)₀₋₂, and O, all of which may be substituted with 1-3 of R¹⁰, or
R²³ is heterocycloalkyl selected from tetrahydrofuiryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
with the proviso for A(OR²²)—R²³ that when R²³ is selected from hydroxy, alkoxy of 1-8 carbon atoms, haloalkoxy of 1-8 carbon atoms, and cycloalkoxy of 3-8 carbon atoms, A is not CH;
R²⁴ and R²⁵ are independently selected from hydrogen, alkyl of 1-6 carbon atoms, haloalkyl of 1-6 carbon atoms, and A-R²³, or
R²⁴ and R²⁵ are independently selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl, all of which may be substituted with 1-3 of R¹⁰, or
R²⁴ and R²⁵ are heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imnidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰, or
R²⁴ and R²⁵ combine, together with the nitrogen atom to which they are attached, to form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, all of which may be substituted with 1-3 of R¹⁰;
R²⁶ is selected from alkyl of 1-6 carbon atoms, haloalkyl of 1-6 carbon atoms, A(OR²²)—R²³, and A-R²³, or
R²⁶ is selected from cyclopropyl, cyclopentyl, cyclohexyl, aryl of 6 or 10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl, all of which may be substituted with 1-3 of R¹⁰, or
R²⁶ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
R²⁷ is selected from hydrogen, alkyl of 1-6 carbon atoms, haloalkyl of 1-6 carbon atoms, and A-R²³, or
R²⁷ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰, or
R²⁷ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
R²⁸ is selected from hydrogen, alkyl of 1-6 carbon atoms, haloalkyl of 1-6 carbon atoms, A-R²³, C(═O)R²⁴, C(═O)OR²⁶, C(═O)NR²⁵R³⁰, S(═O)₂R²⁶, A-C(═O)R²⁴, A-C(═O)OR²⁴, and A-C(═O)NR²⁴R²⁵, or
R²⁸ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl isoquinolinyl, all of which may be substituted with 1-3 of R¹⁰, or
R²⁸ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
R³⁰ is selected from alkyl of 1-6 carbon atoms, haloalkyl of 1-6 carbon atoms, A(OR²²)—R²³, and A-R²³, or
R³⁰ is selected from cyclopropyl, cyclopentyl, cyclohexyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰,
or
R³⁰ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰, or
R²⁵ and R³⁰ combine, together with the nitrogen atom to which they are attached, to form a heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide, thiomorpholinyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, or isoquinolinyl, all of which may be substituted with 1-3 of R¹⁰;
R²⁹ is selected from alkyl of 1-6 carbon atoms, haloalkyl of 1-6 carbon atoms, A-R²³, A-C(═O)R²⁴, A-C(═O)OR²⁴, A-C(═O)NR²⁴R²⁵, A-NR²⁷R²⁸, or
R²⁹ is selected from cyclopropyl, cyclopentyl, cyclohexyl, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, all of which may be substituted with 1-3 of R¹⁰, or
R²⁹ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl or isoquinolinyl, wherein said heterocycloalkyl may be substituted with 1-3 of R¹⁰;
R⁷ is selected from cycloalkyl of 3-8 carbon atoms, aryl of 6-10 carbon atoms, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, all of which may be substituted with 1-3 of R¹⁰,
or
R⁷ is heterocycloalkyl selected from tetrahydrofuryl, pyrrolidinyl, pyrrolinyl, piperidinyl, 1,2-dihydropyridinyl, 1,4-dihydropyridinyl, piperazinyl, morpholinyl, morpholinyl-N-oxide and thiomorpholinyl, wherein said heterocycloalkyl may further be fused with phenyl, thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, and/or wherein one or more of the carbon atoms in said heterocycloalkyl may be oxidized to C(═O), wherein said heterocycloalkyl may be substituted with 1-3 of R¹⁰, A(OR²²)—R²³, A-R²³, A-NR²⁴R²⁵, C(═O)R²⁴, C(═O)OR²⁴, C(═O)NR²⁴R²⁵, S(═O)₂R²⁶, A-C(═O)R²⁴, A-C(═O)OR²⁴, or A-C(═O)NR²⁴R²⁵,
or
R⁷ is selected from hydrogen, nitrile, nitro, hydroxy, alkyl of 1-8 carbon atoms, haloalkyl of 1-8 carbon atoms, alkoxy of 1-8 carbon atoms, haloalkoxy of 1-8 carbon atoms, cycloalkoxy of 3-8 carbon atoms, A-R²³, A(OR²²)—R²³, NR²⁷R²⁸, A-NR²⁷R²⁸, A-Q-R²⁹, Q-R²⁹, Q-A-NR²⁴R²⁵, C(═O)R²⁴, C(═O)OR²⁴, C(═O)NR²⁴R²⁵, A-C(═O)R²⁴, A-C(═O)OR²⁴, and A-C(═O)NR²⁴R²⁵;
and pharmaceutically acceptable salts thereof,
with the proviso that the compound is not 1-(3-chlorophenyl)-4-(dimethylamino)-1,8-naphthyridin-2(1H)-one or 4-amino-1-phenyl-1,8-naphthyridin-2(1H)-one.

3. The compound of claim 1, wherein
R¹ is phenyl, which may be substituted with 1-3 of R¹⁰;
R¹⁰ is selected from nitro, nitrile, hydroxy, halogen, trifluoromethyl, methylcarbonyl, ethylcarbonyl, methyl, ethyl, propyl, isopropyl, butyl, t-butyl, methoxy, ethoxy, propyloxy or isopropyloxy;
R³ is hydrogen,
R⁴ is —NR⁴⁻¹R⁴⁻²;
R⁴⁻¹ is selected from the group consisting of hydrogen, methyl, ethyl, propyl, isopropyl, butyl and t-butyl;
R⁴⁻² is phenyl, wherein said phenyl may be substituted with one to three substituents selected from the group consisting of nitro, nitrile, hydroxy, fluoro, chloro, methylcarbonyl, ethylcarbonyl, methyl, ethyl, propyl, isopropyl, butyl, t-butyl, methoxy, ethoxy, propyloxy or isopropyloxy; or
R⁴⁻¹ and R⁴⁻² form a heterocycloalkyl selected from piperazinyl, morpholinyl and thiomorpholinyl, wherein said heterocycloalkyl may be substituted with one to three substituents selected from the group consisting of nitro, nitrile, hydroxy, fluoro, chloro, methylcarbonyl, ethylcarbonyl, methyl, ethyl, propyl, isopropyl, butyl, t-butyl, methoxy, ethoxy, propyloxy and isopropyloxy,
R⁵ and R⁶ are independently selected from hydrogen, fluoro, chloro, nitrile, nitro, hydroxy, methyl, ethyl, propyl, isopropyl, butyl, t-butyl, trifluoromethyl, methoxy, ethoxy, propyloxy and isopropyloxy;
R⁷ is selected from hydrogen, nitrile, nitro, hydroxy, methyl, ethyl, propyl, isopropyl, butyl, t-butyl, trifluoromethyl, methoxy, ethoxy, propyloxy and isopropyloxy;
and pharmaceutically acceptable salts thereof,
with the proviso that the compound is not 1-(3-chlorophenyl)-4-(dimethylamino)-1,8-naphthyridin-2(1H)-one or 4-amino-1-phenyl-1,8-naphthyridin-2(1H)-one.

4. The compound of claim 1, wherein
R¹ is phenyl, which may be substituted with 1-3 of R¹⁰;
R¹⁰ is selected from fluoro, chloro and trifluoromethyl;
R³ is hydrogen,
R⁴ is —NR⁴⁻¹R⁴⁻²;
R⁴⁻¹ is selected from the group consisting of hydrogen and methyl;
R⁴⁻² is phenyl, wherein said phenyl may be substituted with one or two substituents selected from the group consisting of nitrile, fluoro, chloro, methyl, ethyl, methoxy and ethoxy; or
R⁴⁻¹ and R⁴⁻² form a morpholinyl,
R⁵ and R⁶ are independently selected from hydrogen, fluoro and chloro;
R⁷ is selected from hydrogen, fluoro and chloro;
and pharmaceutically acceptable salts thereof,
with the proviso that the compound is not 1-(3-chlorophenyl)-4-(dimethylamino)-1,8-naphthyridin-2(1H)-one or 4-amino-1-phenyl-1,8-naphthyridin-2(1H)-one.

5. A pharmaceutical composition comprising an effective amount of a compound of claim 1, or a pharmaceutically acceptable salt thereof, in combination with a pharmaceutically acceptable carrier.

6. A pharmaceutical composition comprising a therapeutically effective amount of a compound of claim 1, or a pharmaceutically acceptable salt thereof, in combination with a pharmaceutically acceptable carrier and one or more pharmaceutical agents.

7. The pharmaceutical composition of claim 6, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, anti-obesity agents, HMG CoA reductase inhibitors, nicotinic acid, bile acid sequestrants, bile acid reuptake inhibitors, microsomal triglyceride transport inhibitors, fibric acid derivatives, lipid lowering drugs, ACAT inhibitors, and anti-hypertensive agents.

8. A method of treating diabetes or diabetes-related disorders comprising the step of administering to a subject in need thereof a therapeutically effective amount of a compound of claim 1.

9. The method of claim 8, wherein said diabetes is selected from the group consisting of type 1 diabetes, type 2 diabetes, maturity-onset diabetes of the young, latent autoimmune diabetes adult, and gestational diabetes.

10. A method of treating Syndrome X comprising the step of administering to a subject in need thereof a therapeutically effective amount of a compound of claim 1.

11. A method of treating diabetes-related disorders comprising the step of administering to a subject in need thereof a therapeutically effective amount of a compound of claim 1.

12. The method of claim 11, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

13. A method of treating diabetes comprising the step of administering to a subject in need thereof a therapeutically effective amount of a compound of claim 1 in combination with one or more pharmaceutical agents.

14. The method of claim 13, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, anti-obesity agents, HMG CoA reductase inhibitors, nicotinic acid, bile acid sequestrants, bile acid reuptake inhibitors, microsomal triglyceride transport inhibitors, fibric acid derivatives, lipid lowering drugs, ACAT inhibitors, and anti-hypertensive agents.

15. Compounds according to claim 1 for the treatment and/or prophylaxis of diabetes and diabetes-related disorders.

16. Medicament containing at least one compound according to claim 1 in combination with at least one pharmaceutically acceptable, pharmaceutically safe carrier or excipient.

17. Use of compounds according to claim 1 for manufacturing a medicament for the treatment and/or prophylaxis of diabetes and diabetes-related disorders.

18. Medicaments according to claim 16 for the treatment and/or prophylaxis of diabetes.

19. A process for preparing compounds of the present invention, wherein compounds of formula (I), 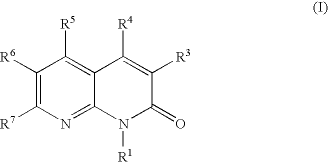
wherein R⁴ represents a leaving group,
is reacted with a compound of formula
HNR⁴⁻¹R⁴⁻²
in the presence of a base.

20. The process of claim 19, wherein the leaving group is selected from halogen, tosylate, mesylate, and triflate.

21. The process of claim 20, wherein the leaving group is chlorine.

22. The process of claim 19, wherein the base is lithium bis(trimethylsilyl)amide
